(19) United States
(12) Patent Application Publication
(10) Pub. No.: US 2008/0021001 A1
(43) Pub. Date: Jan. 24, 2008

(54) Methods For Treating Arthritic Conditions In Dogs

(76) Inventors: Donald Thompson, Annandale, NJ (US); Peter Hanson, Suwanee, GA (US)

Correspondence Address: MERCK AND CO., INC, P O BOX 2000, RAHWAY, NJ, 07065-0907, US

(21) Appl. No.: 11/579,302

(22) PCT Filed: May 3, 2005
(86) PCT No.: PCT/US05/15187

§ 371 Date: Oct. 31, 2006

Related U.S. Application Data
(60) Provisional application No. 60/568,525, filed on May 6, 2004.

(51) Int. Cl. A61K 31/675; A61K 31/66; A61P 19/02

(52) U.S. Cl. 514/89; 514/102; 514/408

(57) ABSTRACT

The present invention relates to a method for eliciting a disease modifying effect on an arthritic condition in a hip or stifle of a canine which comprises administering to the canine a therapeutically effective amount of a bisphosphonate. The present invention also relates to method for eliciting a disease modifying effect on hip dysplasia or stifle instability, the pain associated with hip dysplasia or stifle instability, joint swelling, shallowing of the acetabulum, narrowing of the joint space, subchondral bone sclerosis, preventing osteophyte formation and preventing joint destruction in a canine which comprises administering to the canine a therapeutically effective amount of a bisphosphonate.

### BACKGROUND OF THE INVENTION

Osteoarthritis (OA) is a degenerative joint disease characterized by pain, cartilage loss and joint stiffness. It is a common disease that affects dogs of all ages, but is most prevalent in older animals. It may be a primary disease, the result of general wear and tear, or a secondary disease, the result of injury, infection, non healing fracture or developmental abnormalities.

Hip Dysplasia is a developmental disease of dogs in which a deformity between the head of the femur and the acetabulum creates joint instability allowing excessive movement of the femoral head. This is a common condition in dogs, particularly in large breeds. The exact cause is not known, although there is a genetic component. While the disease may be inherited, the expression of the defect is very largely influenced by factors such as nutrition, growth rates, obesity and exercise.

Initially, hip dysplasia is seen as a loss of joint tightness, allowing the head of the femur excessive movement around the ball of the acetabulum. In the extreme, the joint subluxates. Over time, these abnormal joint interactions create injury and erosion of the articular cartilage covering the ends of the opposing bones. There is pain, joint swelling, a narrowing of the joint space, eburnation (articulation of bone on bone), and structural changes to the joint, including shallowing of the acetabulum, femoral head remodeling and osteophyte development.

The pain associated with this disease can be controlled with varying efficacy by the use of non-steroidal anti-inflammatory drugs. More potent pain relief may be achieved using narcotics. However, these therapies are purely palliative and do not prevent the progression of the osteoarthritis. Eventually, surgery to remove the femoral head, or complete hip replacement, must be considered as the only treatment which is effective in providing pain relief.

Rupture of, or damage to the cruciate ligaments usually occurs due to sudden rotation or hyperextension of the stifle joint during exercise. It commonly involves the cranial cruciate and may be quite painful and involve other injury to the joint. If the ligament is ruptured the resultant joint instability usually leads to degenerative joint changes including joint thickening, meniscal cartilage degeneration, narrowing of the joint space and periarticular osteophyte formation.

If cruciate rupture is diagnosed, surgery to stabilize the joint is indicated. In dogs where surgery is not performed or is not successful, chronic joint instability is likely, leading to development of osteoarthritis. The selection of analgesics that are used to treat hip dysplasia are indicated in dogs with osteoarthritis involving the stifle joint. The analgesics relieve discomfort but do not treat the primary disorder.

### SUMMARY OF THE INVENTION

The present invention relates to a method for eliciting a disease modifying effect on an arthritic condition in a canine which comprises administering to the canine a therapeutically effective amount of a bisphosphonate. The present invention also relates to method for eliciting a disease modifying effect on hip dysplasia, the pain associated with hip dysplasia, joint swelling, shallowing of the acetabulum, subchondral bone sclerosis, preventing osteophyte formation and preventing joint destruction in a canine which comprises administering to the canine a therapeutically effective amount of a bisphosphonate.

### DETAILED DESCRIPTION OF THE INVENTION

The present invention relates to a method for eliciting a disease modifying effect on an arthritic condition in a canine which comprises administering to the canine a therapeutically effective amount of a bisphosphonate.

The present invention relates to a method for treating osteoarthritis resulting from hip dysplasia or stifle instability associated with cruciate ligament damage in a canine which comprises administering to the canine a therapeutically effective amount of a bisphosphonate.

The present invention relates to a method for treating pain associated with hip dysplasia or stifle instability in a canine which comprises administering to the canine a therapeutically effective amount of a bisphosphonate.

The present invention relates to a method for reducing joint swelling in a canine which comprises administering to the canine a therapeutically effective amount of a bisphosphonate.

The present invention relates to a method for preventing shallowing of the acetabulum hip in a canine which comprises administering to the canine a therapeutically effective amount of a bisphosphonate.

The present invention relates to a method for preventing osteophyte formation in a canine which comprises administering to the canine a therapeutically effective amount of a bisphosphonate.

The present invention relates to a method for treating subchondral bone sclerosis in a canine which comprises administering to the canine a therapeutically effective amount of a bisphosphonate.

The present invention relates to a method for preventing joint deterioration in a canine which comprises administering to the canine a therapeutically effective amount of a bisphosphonate.

The present invention relates to a method for eliciting a disease modifying effect on an arthritic condition in a canine which comprises administering to the canine a therapeutically effective amount of a bisphosphonate and a therapeutically effective amount of a nonsteroidal anti-inflammatory drug. The present invention further relates to a pharmaceutical composition comprising a bisphosphonate and a nonsteroidal anti-inflammatory drug.

“Bisphosphonate” includes, but is not limited to, compounds of the chemical formula 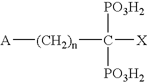
wherein n is an integer from 0 to 7 and wherein A and X are independently selected from the group consisting of H, OH, halogen, NH₂, SH, phenyl, C1-C30 alkyl, C3-C30 branched or cycloalkyl, bicyclic ring structure containing two or three N, C1-C30 substituted alkyl, C1-C10 alkyl substituted NH₂, C3-C10 branched or cycloalkyl substituted NH₂, C1-C10 dialkyl substituted NH₂, C1-C10 alkoxy, C1-C10 alkyl substituted thio, thiophenyl, halophenylthio, C1-C10 alkyl substituted phenyl, pyridyl, furanyl, pyrrolidinyl, imidazolyl, imidazopyridinyl, and benzyl, such that both A and X are not selected from H or OH when n is 0; or A and X are taken together with the carbon atom or atoms to which they are attached to form a C3-C10 ring.

In the foregoing chemical formula, the alkyl groups can be straight, branched, or cyclic, provided sufficient atoms are selected for the chemical formula. The C1-C30 substituted alkyl can include a wide variety of substituents, nonlimiting examples which include those selected from the group consisting of phenyl, pyridyl, furanyl, pyrrolidinyl, imidazonyl, NH₂, C1-C10 alkyl or dialkyl substituted NH₂, OH, SH, and C1-C10 alkoxy.

The foregoing chemical formula is also intended to encompass complex carbocyclic, aromatic and hetero atom structures for the A and/or X substituents, non-limiting examples of which include naphthyl, quinolyl, isoquinolyl, adamantyl, and chlorophenylthio.

Pharmaceutically acceptable salts and derivatives of the bisphosphonates are also useful herein. Non-limiting examples of salts include those selected from the group consisting alkali metal, alkaline metal, ammonium, and mono-, di-, tri-, or tetra-C1-C30-alkyl-substituted ammonium. Preferred salts are those selected from the group consisting of sodium, potassium, calcium, magnesium, and ammonium salts. More preferred are sodium salts. Non-limiting examples of derivatives include those selected from the group consisting of esters, hydrates, and amides.

It should be noted that the terms “bisphosphonate” and “bisphosphonates”, as used herein in referring to the therapeutic agents of the present invention are meant to also encompass diphosphonates, bisphosphonic acids, and diphosphonic acids, as well as salts and derivatives of these materials. The use of a specific nomenclature in referring to the bisphosphonate or bisphosphonates is not meant to limit the scope of the present invention, unless specifically indicated. Because of the mixed nomenclature currently in use by those of ordinary skill in the art, reference to a specific weight or percentage of a bisphosphonate compound in the present invention is on an acid active weight basis, unless indicated otherwise herein. For example, the phrase “about 5 mg of a bone resorption inhibiting bisphosphonate selected from the group consisting of alendronate, pharmaceutically acceptable salts thereof, and mixtures thereof, on an alendronic acid active weight basis” means that the amount of the bisphosphonate compound selected is calculated based on 5 mg of alendronic acid.

Non-limiting examples of bisphosphonates useful herein include the following:

Alendronate, which is also known as alendronic acid, alendronate sodium or alendronate monosodium trihydrate, 4-amino-1-hydroxybutylidene-1,1-bisphosphonic acid and 4-amino-1-hydroxybutylidene-1,1-bisphosphonic acid monosodium trihydrate, are described in U.S. Pat. No. 4,922,007, to Kieczykowski et al., issued May 1, 1990; U.S. Pat. No. 5,019,651, to Kieczykowski et al., issued May 28, 1991; U.S. Pat. No. 5,510,517, to Dauer et al., issued Apr. 23, 1996; U.S. Pat. No. 5,648,491, to Dauer et al., issued Jul. 15, 1997, all of which are incorporated by reference herein in their entirety.

Cycloheptylaminomethylene-1,1-bisphosphonic acid, YM 175, Yamanouchi (incadronate, formerly known as cimadronate), as described in U.S. Pat. No. 4,970,335, to Isomura et al., issued Nov. 13, 1990, which is incorporated by reference herein in its entirety.

1,1-dichloromethylene-1,1-diphosphonic acid (clodronic acid), and the disodium salt (clodronate, Procter and Gamble), are described in Belgium Patent 672,205 (1966) and J. Org. Chem 32, 4111 (1967), both of which are incorporated by reference herein in their entirety.

1-hydroxy-3-(1-pyrrolidinyl)-propylidene-1,1-bisphosphonic acid (EB-1053).

1-hydroxyethane-1,1-diphosphonic acid (etidronic acid).

1-hydroxy-3-(N-methyl-N-pentylamino)propylidene-1,1-bisphosphonic acid, also known as BM-210955, Boehringer-Mannheim (ibandronate), is described in U.S. Pat. No. 4,927,814, issued May 22, 1990, which is incorporated by reference herein in its entirety.

1-hydroxy-2-imidazo-(1,2-a)pyridin-3-yethylidene (minodronate).

6-amino-1-hydroxyhexylidene-1,1-bisphosphonic acid (neridronate).

3-(dimethylamino)-1-hydroxypropylidene-1,1-bisphosphonic acid (olpadronate).

3-amino-1-hydroxypropylidene-1,1-bisphosphonic acid (pamidronate).

[2-(2-pyridinyl)ethylidene]-1,1-bisphosphonic acid (piridronate) is described in U.S. Pat. No. 4,761,406, which is incorporated by reference in its entirety.

1-hydroxy-2-(3-pyridinyl)-ethylidene-1,1-bisphosphonic acid (risedronate).

(4-chlorophenyl)thiomethane-1,1-disphosphonic acid (tiludronate) as described in U.S. Pat. No. 4,876,248, to Breliere et al., Oct. 24, 1989, which is incorporated by reference herein in its entirety.

1-hydroxy-2-(1H-imidazol-1-yl)ethylidene-1,1-bisphosphonic acid (zoledronate).

Non-limiting examples of bisphosphonates include alendronate, cimadronate, clodronate, etidronate, ibandronate, incadronate, minodronate, neridronate, olpadronate, pamidronate, piridronate, risedronate, tiludronate, and zolendronate, and pharmaceutically acceptable salts and esters thereof. A particularly preferred bisphosphonate is alendronate, especially a sodium, potassium, calcium, magnesium or ammonium salt of alendronic acid. Exemplifying the preferred bisphosphonate is a sodium salt of alendronic acid, especially a hydrated sodium salt of alendronic acid. The salt can be hydrated with a whole number of moles of water or non whole numbers of moles of water. Further exemplifying the preferred bisphosphonate is a hydrated sodium salt of alendronic acid, especially when the hydrated salt is alendronate monosodium trihydrate.

It is recognized that mixtures of two or more of the bisphosphonate actives can be utilized.

Definitions

“Arthritic condition” or “arthritic conditions” refers to a disease wherein inflammatory lesions are confined to the joints or any inflammatory conditions of the joints.

“Joint Swelling” refers to an expansion of the external circumference of the joint due to effusion into the joint space or to external thickening of the joint capsule and surrounding structures.

“Shallowing of the acetabulum” refers to a remodeling of the shape of the acetabulum so that the depth of the cup into which the head of the femur normally opposes is reduced and the cup shape is flattened.

“Narrowing of the joint space” refers to apparent reduction in the distance between the opposing bones which articulate within a joint. It is the result of the reduction in thickness of cartilage covering the articular surface of the bones, this reduction permitting the bones to be in closer proximity to each other than in a normal joint.

“Subchondral bone sclerosis” as used herein means the increase in bone density and volume in the subchondral region.

“Osteophyte” as used herein refer to newly formed bony structures located at the joint margins, and their occurrence is strongly associated with the late stage of OA progression. The current hypothesis is that osteophytes originate from activated periosteum leading to new cartilaginous outgrowths that eventually turns into bone by the process of endochondral bone formation.

“Joint destruction” as used herein refers to the destruction of articular cartilage.

The term “disease modifying effect” refers to an agent that can slow, retard or prevent the progression of a disease. For example, in the case of osteoarthritis, a disease modifying effect could include slowing the loss of cartilage and preventing osteophyte formation.

A “Nonsteroidal anti-inflammatory drug (NSAID)” refers to non steroidal therapeutics that limit the formation of inflammation. Nonlimiting examples of NSAIDS include, but are not limited to, carprofen, etodolac, ibuprofen, ketoprofen, meloxicam, naproxen and selective cyclooxygenase-2 inhibitors (COX-2 inhibitors). Nonlimiting examples of COX-2 inhibitors include: celecoxib, deracoxib, etoricoxib, firocoxib, lumaricoxib, parecoxib, rofecoxib, and valdecoxib.

The term “composition” as used herein is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The terms “treating” or “treatment” of a disease as used herein includes: preventing the disease, i.e. causing the clinical symptoms of the disease not to develop in a canine that may be exposed to or predisposed to the disease but does not yet experience or display symptoms of the disease; inhibiting the disease, i.e., arresting or reducing the development of the disease or its clinical symptoms; or relieving the disease, i.e., causing regression of the disease or its clinical symptoms.

As used herein, the term “pharmaceutically acceptable salts” includes the conventional non-toxic salts of the compounds of this invention as formed inorganic or organic acids. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. The preparation of the pharmaceutically acceptable salts described above and other typical pharmaceutically acceptable salts is more fully described by Berg et al., “Pharmaceutical Salts,” J. Pharm. Sci., 1977:66:1-19, hereby incorporated by reference. The pharmaceutically acceptable salts of the compounds of this invention can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

Utilities

The compositions and methods of the present invention are useful for eliciting a disease modifying effect on arthritic conditions, especially for eliciting a disease modifying effect on osteoarthritis and hip dysplasia in canines, including the treatment of pain associated with hip dysplasia, reduction of joint swelling, and prevention of the shallowing of the acetabulum, subchondral bone resorption, osteophyte formation and ultimately joint deterioration/destruction.

The methods of the present invention have an unexpected disease modifying effect in the treatment of arthritic conditions in canines.

The compositions of the present invention can be administered in such oral dosage forms as tablets, capsules (each of which includes sustained release or timed release formulations), pills, powders, granules, elixirs, pastes, tinctures, sterile solutions or suspensions, syrups, flavored treats and emulsions. Likewise, it may also be administered in intravenous (bolus or infusion), intraperitoneal, topical (e.g., ocular eyedrop), intranasal, inhaled, subcutaneous, intramuscular or transdermal (e.g., patch) form, metered aerosol or liquid sprays, drops, ampoules, auto-injector devices or suppositories all using forms well known to those of ordinary skill in the pharmaceutical arts. An effective but non-toxic amount of the compositions desired can be employed. The compositions are intended for oral, parenteral, intranasal, sublingual, or rectal administration, or for administration by inhalation or insufflation. Formulation of the compositions according to the invention can conveniently be effected by methods known from the art, for example, as described in Remington's Pharmaceutical Sciences, 17^{th} ed., 1995.

The dosage regimen utilizing the compositions of the present invention is selected in accordance with a variety of factors including type, species, age, weight, sex and medical condition of the subject; the severity of the condition to be treated; the route of administration; the renal and hepatic function of the subject; and the particular compound or salt thereof employed. An ordinarily skilled veterinarian or clinician can readily determine and prescribe the effective amount of the drug required to prevent, counter or arrest the progress of the condition.

Advantageously, the compounds of the present invention may be administered in a single quarterly, monthly, weekly or daily dose, or the total daily dosage may be administered in divided doses of two, three or four times daily. Furthermore, the compound of the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

The dose may be administered in a single daily dose or the total daily dosage may be administered in divided doses of two, three or four times daily. Furthermore, based on the properties of the individual compound selected for administration, the dose may be administered less frequently, e.g., weekly, twice weekly, monthly, etc. The unit dosage will, of course, be correspondingly larger for the less frequent administration.

The precise dosage of the bisphosphonate will vary with the dosing schedule, the oral potency of the particular bisphosphonate chosen, the age, size, sex and condition of the canine, the nature and severity of the disorder to be treated, and other relevant medical and physical factors. For canines, an effective oral dose of bisphosphonate is typically from about 1.5 to about 20,000 μg/kg body weight and preferably about 10 to about 10,000 μg/kg of body weight.

In alternative dosing regimens, the bisphosphonate can be administered at intervals other than daily, for example once-weekly dosing, twice-weekly dosing, biweekly dosing, and twice-monthly dosing. In a once weekly dosing regimen, alendronate monosodium trihydrate would be administered at dosages of about 2.5 mg/week to about 280 mg/week. Nonlimiting examples of doses include 140 mg/week and 280 mg/week. The bisphosphonates may also be administered monthly, ever six months, yearly or even less frequently, see WO 01/97788 (published Dec. 27, 2001) and WO 01/89494 (published Nov. 29, 2001).

According to a further aspect of the present invention, it may be desirable to treat any of the aforementioned conditions with a combination of a bisphosphonate and one or more other pharmacologically active agents suitable for the treatment of the specific condition. The bisphosphonate and the other pharmacologically active agent(s) may be administered to a subject simultaneously, sequentially or in combination. For example, the present compound may be employed directly in combination with the other active agent(s), or it may be administered prior, concurrent or subsequent to the administration of the other active agent(s). In general, the currently available dosage forms of the known therapeutic agents for use in such combinations will be suitable.

The compositions and methods of the present invention are administered and carried out until the desired therapeutic effect is achieved.

The identification of a bisphosphonate which is able to have utility in the present invention may be readily determined without undue experimentation by methodology well known in the art, such as the assay described herein.

### Assay

Materials and Methods

Osteoarthritis model and treatment—All procedures were carried out according to the Institutional Animal Care and Use Committee Guide in Merck Research Labs. Ninety-five 20-week old male Sprague-Dawley rats (Taconic, NJ) were used following experiments. Osteoarthritis (OA) model was surgically induced in 20-wk-old male rat knee joints or in 7-10 month old male NZ White rabbits. Briefly, the animals were anesthetized by isoflurane. The right knee joint was shaved, disinfected with iodine, and exposed though the medial parapatellar approach. The patella was dislocated laterally and the knee placed in full flexion. All operation procedures were performed using a surgical loupe. Anterior cruciate ligament (ACL) was transected with micro-scissors. To confirm complete transection of ACL, Lachman test was performed. After surgery, the joint surface was washed with sterile saline solution, and both capsule and skin were sutured using Vicryl 4-0 (Ethicon, Edinburgh, UK), absorbable suture and monofilament 4-0 Nylon threads (Ethicon, Edinburgh, UK). In Sham operation, the wound was closed by layers after subluxation of patella and saline washing. Buprenorphine hydrochloride (0.1 mg/kg) (Reckitt & Colman Products Ltd., Hull, England) was given as an analgesic. Animals were allowed to move freely in the soft bedding plastic cages.

A test compound was administered by either subcutaneous injection or orally dosing. Drug was dosed prior to the surgery in the prevention mode. In treatment mode, drug was dosed 1 or 2 weeks post-surgery. Endpoints were histological analysis, histomorphometry and evaluation of serum markers. In all studies, the animals were always included the following groups: ACL transection with vehicle, ACLT with a low and a higher doses of the drug, sham operation with vehicle, and sham operation with the high dose of the drug. Animals were sacrificed on 2- and 10-wk post-surgery with CO₂. In both time points, rats were injected 10-mg/kg calcein 3 days before the necropsy. In a separate study, the same groups of animals received either sham- or ACLT-operation and with or without drug treatment were used for TGF-β assay. These animals were sacrificed on 2-wk post-surgery.

Gross morphology, Tissue preparation and histology—After the disarticulation of the right joint, both femur and tibia were carefully cleaned free of muscles, and fixed in 4% paraformaldehyde (Fisher Scientific, NJ) in phosphate buffer saline (PBS) for 24 hrs. Gross appearance of the distal femur was taken by digital camera (DIX, Nikon, Japan) with 1:4 Nikkor lens (Nikon, Japan) to evaluate osteophyte formation. Tibia was then cut in a half at the center of articular surface along with medial collateral ligament in frontal section with band saw (EXAKT Technologies, Inc, Norderstedt, Germany). Anterior parts were re-immersed in 4% paraformaldehyde for another 24 hrs for paraffin embedding. Posterior parts were changed into 70% ethanol, and then embedded in methylmethacrylate. Sections at 5 μm thick were stained Masson's trichrome staining as described previously, see Gruber, H. E., G. J. Marshall, L. M. Nolasco, M. E. Kirchen, and D. L. Rimoin, 1988, “Alkaline and acid phosphatase demonstration in human bone and cartilage: effects of fixation interval and methacrylate embedments,” Stain Technol. 63:299-306 and Yamamoto, M., J. E. Fisher, M. Gentile, J. G. Seedor, C. T. Leu, S. B. Rodan, and G. A. Rodan, 1998, “The integrin ligand echistatin prevents bone loss in ovariectomized mice and rats” Endocrinology. 139:1411-9. Specimens were labeled with randomly assigned identification numbers to blind the investigator to the group designation during subsequent measurements.

For paraffin embedding, tissues were decalcified in 0.5 M ethylenedinitrilo-tetra acetic acid solution (pH 7.6, Fisher Scientific, NJ) for 7 to 10 days, then treated with a graded ethanol series, followed by xylene, prior to embedding into paraffin wax (Fisher Scientific, NJ) as previously described, see Nakase, T., K. Takaoka, K. Hirakawa, S. Hirota, T. Takemura, H. Onoue, K. Takebayashi, Y. Kitamura, and S. Nomura, 1994, “Alterations in the expression of osteonectin, osteopontin and osteocalcin mRNAs during the development of skeletal tissues in vivo,” Bone Miner. 26:109-22 and Hayami, T., N. Endo, K. Tokunaga, H. Yamagiwa, H. Hatano, M. Uchida, and H. E. Takahashi, 2000, “Spatiotemporal change of rat collagenase (MMP-13) mRNA expression in the development of the rat femoral neck,” J Bone Miner Metab. 18:185-93.

Paraffin embedded specimen was sectioned and examined by histological analysis and immunohistochemistry. Paraffin sections were stained with toluidine blue-O (0.2% toluidine blue-O/0.1M sodium acetate buffer, pH 4.0) for proteoglycan content. Occasionally, sections were also stained with tartrate resistant acid phosphatase (TRAP) stain for osteoclast localization, as previously described, see Nakamura, Y., A. Yamaguchi, T. Ikeda, and S. Yoshiki, 1991, “Acid phosphatase activity is detected preferentially in the osteoclastic lineage by pre-treatment with cyanuric chloride,” J Histochem Cytochem. 39:1415-20.

Histopathological scores (modified Mankin score)—Semi-quantitative histopathological grading was performed according to a modified Mankin scoring system, which is a well established grading system in OA research, with some modifications, see Cake, M. A., R. A. Read, B. Guillou, and P. Ghosh, 2000, “Modification of articular cartilage and subchondral bone pathology in an ovine meniscectomy model of osteoarthritis by avocado and soya unsaponifiables (ASU),” Osteoarthritis Cartilage. 8:404-11; Little, C., S. Smith, P. Ghosh, and C. Bellenger, 1997, “Histomorphological and immunohistochemical evaluation of joint changes in a model of osteoarthritis induced by lateral meniscectomy in sheep,” J Rheumatol. 24:2199-209; Wenz, W., S. J. Breusch, J. Graf, and U. Stratmann, 2000, “Ultrastructural findings after intraarticular application of hyaluronan in a canine model of arthropathy,” J Orthop Res. 18:604-12.

Mankin score normally consists of five subcategories, including structure, chondrocyte number, chondrocyte clustering, proteoglycan content (stainability for toluidine blue-O), and subchondral plate and/or tidemark change including vascular invasion in cartilage. Since vascular invasion into cartilage was independently evaluated using Masson's trichrome staining, we omitted this category in the Mankin score. Three sections 100 μm apart were measured in each sample. Total possible score is 26 and scoring was done by a single observer with blinded according to a five-point scale (Cake et al. 2000). Low total score are consistent with minor degenerative cartilaginous lesions, whereas high total score indicative of more pronounced cartilaginous regions. In toluidine blue-O staining stainability, we use the terminology as previously described (Little, et al. 1997), “mild” was used when there was decreased toluidine blue-O staining with intact articular surface, “moderate” when there was decreased toluidine blue-O staining in association with surface fibrillation and clefts extending to but not below the middle zone, and “severe” when cartilage was lost down to the level of the calcified cartilage.

Bone histomorphometry—For quantification of the histological parameters, we used Image Pro plus (version 4, Media Cybernetics, MD) image analysis program. Images of articular cartilage and subchondral bone were examined using a Olympus fluorescence microscope (BX51, Japan) with ×4 objective lens and were recorded using a CCD/RGB color video camera (RT Slider SPOT, Diagnostic instrument. Inc., MI).

Histomorphometric measurements of both medial and lateral tibial plateaux were determined in two separate sections per knee joint, spaced 100 μm apart. Since subchondral region has been reported that affected in OA development, we developed a macro to measure subchondral bone volume per tissue area. Two areas from either medial or lateral tibial plateau, 600 μm depth×800 μm width, were measured with the center of the tibial plateau being semi-automatically determined according to the width of the tibial surface. To consistently place the area to be measured, the top of the rectangle always horizontally aligned along the surface of articular cartilage and its sides vertically aligned along the center line of the tibia. The data from two areas were combined for the medial or lateral tibial plateau, and measurements of 6 knees per group were averaged in each group.

Trabecular bone volume (BV/TV: percentage of endosteal bone and marrow compartment occupied by osteoid and mineralized bone) in subchondral region was measured by histomorphometric methods that complied with the nomenclature and were calculated according to the ASBMR guidelines, see Parfitt, A. M., M. K. Drezner, F. H. Glorieux, J. A. Kanis, H. Malluche, P. J. Meunier, S. M. Ott, and R. R. Recker, 1987, “Bone histomorphometry: standardization of nomenclature, symbols, and units,” Report of the ASBMR Histomorphometry Nomenclature Committee. J Bone Miner Res. 2:595-610. To detect active bone remodeling surfaces in the subchondral region, we also injected the rats with calcein (10 mg/kg) 3 days before necropsy. Labeled mineralized surfaces in the plastic sections can be viewed using the same Olympus fluorescence microscope as described above.

Vascular invasion into calcified cartilage—Vascular invasion into the calcified cartilage was quantified by counting the number of times the calcified cartilage contacted by subchondral marrow space as previously described, see O'Connor, K. M., 1997, “Unweighting accelerates tidemark advancement in articular cartilage at the knee joint of rats,” J Bone Miner Res. 12:580-9. The results from two sections, spaced 100 μm apart were measured.

Osteoclast score—TRAP positive cells were counted in calcified cartilage and osteophyte regions. The number of TRAP positive cells from two sections in each sample spaced 100 μm apart were measured and then averaged from 6 knees per group.

Osteophytes score and area—Osteophytes were defined as outgrowth of the bone and cartilage occurring at the joint margins in the tibial plateau. To evaluate incident of osteophyte formation (osteophyte score), total osteophyte number from 5 sections including 3 paraffin (anterior part of tibia) and 2 plastic sections (posterior part of tibia) at 100 μm apart, were evaluated from each knee joint. Surface area of each osteophyte was manually determined in Masson's trichrome stained sections using image pro analysis. Two sections, each section is 100 μm apart, were evaluated.

Serum and Urinary levels of COMP, CTX-I and CTX-II—Blood was obtained from cardiac puncture at each necropsy, 2- and 10-wk post-surgery. Serum samples were collected, and frozen in aliquots −70° C. Serum cartilage oligomeric matrix protein (COMP) were determined by AnaMar Medical AB (Uppsala, Sweden) using a modified enzyme-liked immunosorbent assay as previously described, see Larsson, E., A. Mussener, D. Heinegard, L. Klareskog, and T. Saxne, 1997, “Increased serum levels of cartilage oligomeric matrix protein and bone sialoprotein in rats with collagen arthritis,” Br J Rheumatol. 36:1258-61 and Saxne, T., and D. Heinegard, 1992, “Cartilage oligomeric matrix protein: a novel marker of cartilage turnover detectable in synovial fluid and blood,” Br J Rheumatol. 31:583-91.All determinations were done in duplicate.

Twenty-four-hour urine samples were collected from the individual animal's metabolic cages at 2 wk-post surgery. Samples were centrifuged and frozen in aliquots at −70° C. Assays for bone related degradation product from C-terminal telopeptide of type I collagen (CTX-I/Ratlaps, Nordic Bioscience Diagnostics, Denmark) were performed in our laboratory according to the manufacturer's instruction. Assays for cartilage related C-terminal telopeptide of type II collagen (CTX-II/CartiLaps) were performed by Nordic Bioscience Diagnostics, Denmark. Urinary creatinine determination was measured in each sample as a test for normal urinary output. CTX-I and CTX-II values were reported after normalized to the creatine concentration in the same sample.

Immunohistochemistry—Tissue sections were deparaffinized in xylene, hydrated in graded ethanol, then treated with 500 U/ml testicular hyaluronidase (Sigma, MO) at 37° C. for 20 min. Tissue sections were then incubated with using either anti-rat CD31 mAb (Endogen, MA), or anti-activated TGF-β, which recognizes only active form of TGF-β1, 2, and 3 (R&D) as described previously, see Fernandez, T., S. Amoroso, S. Sharpe, G. M. Jones, V. Bliskovski, A. Kovalchuk, L. M. Wakefield, S. J. Kim, M. Potter, and J. J. Letterio, 2002, “Disruption of transforming growth factor beta signaling by a novel ligand-dependent mechanism,” J Exp Med. 195:1247-55, anti-MMP-13 Ab, anti-MMP-9 Ab for over night at 4° C. as described previously, see Hayami, T., H. Funaki, K. Yaoeda, K. Mitui, H. Yamagiwa, K. Tokunaga, H. Hatano, J. Kondo, Y. Hiraki, T. Yamamoto, L. T. Duong, and N. Endo, 2003, “Expression of the cartilage-derived anti-angiogenic factor Chondromodulin-I decreases in the early stage of experimental osteoarthritis,” J. Rheumatol. (in press). In CD31 immunostaining, after rinsing in PBS with 0.3% Tween 20, they were incubated with biotin-conjugated anti-mouse Ab (LSAB2 kit, Dako, CA) for 10 min and followed with alkaline phosphatase-conjugated streptavidin for 10 min (Dako, CA). These sections were rinsed with PBS, and developed using fast red substrate system (Dako, CA) for 5 min and counterstained with hematoxyline. Double-labeled immuno-histochemical stainings with MMP-9/MMP-13 and TGF-β Abs were performed as previously described, see Hayami, T., H. Funaki, K. Yaoeda, K. Mitui, H. Yamagiwa, K. Tokunaga, H. Hatano, J. Kondo, Y. Hiraki, T. Yamamoto, L. T. Duong, and N. Endo, 2003, “Expression of the cartilage-derived anti-angiogenic factor Chondromodulin-I decreases in the early stage of experimental osteoarthritis,” J. Rheumatol. (in press). Briefly, tissue sections were incubated with TGF-β mAb, followed by AP-conjugated anti-mouse Ab, and developed to blue color with AP blue (Vector Laboratories, CA USA). They were washed twice with PBS with 0.3% Tween 20 for 1 hr, incubated with anti-MMP-9 or MMP-13 polyclonal Ab, followed by HRP-anti-rabbit Ab (DAKO, CA), and developed to brown color by 0.5 mg/ml 3,3′-diaminobenzidine tetrahydrochloride. As negative controls, the same procedures were carried out either without primary Ab or with mouse mAb IgG instead of primary antibody.

Mink Lung epithelial growth inhibition assay for TGF-β in supernatant from tibial plateaux/patellae organ culture—Patellae and tibial plateau were isolated from either ACLT- or sham operated joints with or without drug treatment. After disarticulation and dissection of the patellae, tibiae were carefully removed of soft tissue. Articular cartilage and subchondral bone tissue were cut by a bone saw (Buehler Isomet, IL) at 480 μm thickness from the articular surface. Dissected patellae and tibial plateaux were transferred to 24 well culture dishes, washed with 0.1% BSA α-MEM for 3 times, then incubated in same media at 37° C. under 5% CO₂. Supernatant after 12 hrs incubation was collected and frozen at −70° C. Active TGF-β was measured as described previously by using the mink lung epithelial cell bioassay, see Docagne, F., N. Colloc'h, V. Bougueret, M. Page, J. Paput, M. Tripier, P. Dutartre, E. T. MacKenzie, A. Buisson, S. Komesli, and D. Vivien, 2001, “A soluble transforming growth factor-beta (TGF-beta) type I receptor mimics TGF-beta responses,” J Biol Chem. 276:46243-50. Briefly, mink lung cells (Mv1Lu, ATCC, MD) were plated at 10,000 cells/well in 96-well CytoStar scintillating microplates (Amersham, NJ) in E-MEM, 10% FBS containing sodium pyruvate and non-essential amino acids. After 24 hrs, TGF-β1 was diluted in α-MEM (1:4) as final concentration and 50 μl was added to duplicate wells as a control, followed by adding condition media (50 μl/well). After 20 hrs, [¹⁴C-methyl]-thymidine was added to each well to a final dilution of 0.5 μCi/ml. Plates were counted after 4 hr and 24 hr. Data reported was from the 24 hr-time point.

Statistical analysis—Statistical comparisons were generated using Statview (SAS Institute Inc., NC). All data in tables 1-3 were shown as means ±SD. Results are expressed as mean ±SEM. Significance of difference between groups was evaluated with a one-way analysis of variance (ANOVA) to analyze variance across treatment groups, and Fisher's analysis of least significant difference (Fisher's PLSD) to compare treatment group means except where indicated. Difference in values was considered significant when p value was <0.05.

### EXAMPLES

The following examples further describe and demonstrate embodiments within the scope of the present invention. The examples are given solely for the purpose of illustration and are not to be construed as limitations of the present invention as many variations thereof are possible without departing from the spirit and scope of the invention.

Pharmaceutical Tablet Compositions

Tablets are prepared using standard mixing and formation techniques as described in U.S. Pat. No. 5,358,941, to Bechard et al., issued Oct. 25, 1994, which is incorporated by reference herein in its entirety.

Tablets containing about 6.5 mg of alendronate monosodium trihydrate, on an alendronic acid active basis are prepared using the following relative weights of ingredients.

| Ingredient | Per 84 mg Tablet | | Per 4000 Tablets | |
|---|---|---|---|---|
| Alendronate Monosodium | 6.5255 | mg | 26.10 | g |
| Trihydrate | | | | |
| Anhydrous Lactose, NF | 35.66 | mg | 142.64 | g |
| Microcrystalline Cellulose, NF | 40.0 | mg | 160.0 | g |
| Magnesium Stearate, NF | 0.5 | mg | 20 | g |
| Croscarmellose Sodium, NF | 1.0 | mg | 4.0 | g |

The resulting tablets are useful for administration in accordance with the methods of the present invention for inhibiting, i.e. treating or reducing the risk of osteoarthritis associated with hip dysplasia or cruciate ligament damage in a canine in need thereof.

Similarly, tablets comprising other relative weights of alendronate, on an alendronic acid active weight basis are prepared. Also, tablets containing other bisphosphonates at appropriate active levels are similarly prepared: e.g., cimadronate, ibandronate, neridronate, olpandronate, risedronate, piridronate, pamidronate, zolendronate, and pharmaceutically acceptable salts thereof. In addition, tablets containing combinations of bisphosphonates are similarly prepared.

Non Beef Based Chewable Treats

The resulting chewable treats are useful for administration in accordance with the methods of the present invention for inhibiting, i.e. treating or reducing the risk of, osteoarthritis lesions in a dog in need thereof.

Similarly, chewable treats comprising other relative weights of alendronate, on an alendronic acid active weight basis are prepared. Also, chewable treats containing other bisphosphonates at appropriate active levels are similarly prepared: e.g., cimadronate, ibandronate, neridronate, olpandronate, risedronate, piridronate, pamidronate, zolendronate, and pharmaceutically acceptable salts thereof. In addition, chewable treats containing combinations of bisphosphonates are similarly prepared.

| Ingredient | Percent W/W |
|---|---|
| Alendronate Monosodium Trihydrate | 2 |
| Soy Protein fines | 42 |
| Propylene glycol | 6 |
| Water | 22 |
| Artificial beef flavor | 2 |
| Corn starch | 25 |
| Citric Acid | 1 |

Suspensions

The resulting suspensions are useful for administration in accordance with the methods of the present invention for inhibiting, i.e. treating or reducing the risk of, osteoarthritis lesions in a mammal in need thereof.

Similarly, suspensions comprising other relative weights of alendronate, on an alendronic acid active weight basis are prepared. Also, suspensions containing other bisphosphonates at appropriate active levels are similarly prepared: e.g., cimadronate, ibandronate, neridronate, olpandronate, risedronate, piridronate, pamidronate, zolendronate, and pharmaceutically acceptable salts thereof. In addition, suspensions containing combinations of bisphosphonates are similarly prepared.

| Ingredient | Percent W/W |
|---|---|
| Alendronate Monosodium Trihydrate | 1.3% w/w |
| Colloidal Silicon dioxide | 3.0 |
| Alpha-tocopherol | 0.2 |
| Fish Oil | 95.5 |

Solutions

The resulting solutions are useful for administration in accordance with the methods of the present invention for inhibiting, i.e. treating or reducing the risk of, osteoarthritis lesions in a mammal in need thereof.

Similarly, solutions comprising other relative weights of alendronate, on an alendronic acid active weight basis are prepared. Also, solutions containing other bisphosphonates at appropriate active levels are similarly prepared: e.g., cimadronate, ibandronate, neridronate, olpandronate, risedronate, piridronate, pamidronate, zolendronate, and pharmaceutically acceptable salts thereof. In addition, solutions containing combinations of bisphosphonates are similarly prepared.

| Ingredient | Percent W/V |
|---|---|
| Alendronate Monosodium Trihydrate | 1.3% w/v |
| Citric Acid | 1.0 |
| Sodium Citrate | 0.5 |
| Butterscotch Flavor | 0.2 |
| Purified Water | 97.0 |

Ointments

The resulting ointments are useful for administration in accordance with the methods of the present invention for inhibiting, i.e. treating or reducing the risk of, osteoarthritis lesions in a mammal in need thereof.

Similarly, ointments comprising other relative weights of alendronate, on an alendronic acid active weight basis are prepared. Also, ointments containing other bisphosphonates at appropriate active levels are similarly prepared: e.g., cimadronate, ibandronate, neridronate, olpandronate, risedronate, piridronate, pamidronate, zolendronate, and pharmaceutically acceptable salts thereof. In addition, ointments containing combinations of bisphosphonates are similarly prepared.

| Ingredient | Percent W/W |
|---|---|
| Alendronate Monosodium Trihydrate | 1.3% w/w |
| Lecithin | 3.0 |
| Malt Syrup | 45.0 |
| White Petrolatum | 50.7 |

Gels

The resulting gels are useful for administration in accordance with the methods of the present invention for inhibiting, i.e. treating or reducing the risk of, osteoarthritis lesions in a mammal in need thereof.

Similarly, gels comprising other relative weights of alendronate, on an alendronic acid active weight basis are prepared. Also, gels containing other bisphosphonates at appropriate active levels are similarly prepared: e.g., cimadronate, ibandronate, neridronate, olpandronate, risedronate, piridronate, pamidronate, zolendronate, and pharmaceutically acceptable salts thereof. In addition, gels containing combinations of bisphosphonates are similarly prepared.

| Ingredient | Percent W/W |
|---|---|
| Alendronate Monosodium Trihydrate | 1.3% w/w |
| Citric Acid | 1.0 |
| Sodium Citrate | 0.5 |
| Poloxamer | 20.0 |
| Propylene Glycol | 20.0 |
| Benzyl Alcohol | 2.0 |
| Purified Water | 57.0 |

Pastes

The resulting pastes are useful for administration in accordance with the methods of the present invention for inhibiting, i.e. treating or reducing the risk of, osteoarthritis lesions in a mammal in need thereof.

Similarly, pastes comprising other relative weights of alendronate, on an alendronic acid active weight basis are prepared. Also, pastes containing other bisphosphonates at appropriate active levels are similarly prepared: e.g., cimadronate, ibandronate, neridronate, olpandronate, risedronate, piridronate, pamidronate, zolendronate, and pharmaceutically acceptable salts thereof. In addition, pastes containing combinations of bisphosphonates are similarly prepared.

| Ingredient | Percent W/W |
|---|---|
| Alendronate Monosodium Trihydrate | 1.3% w/w |
| Sodium Carboxymethylcellulose | 2.0 |
| Magnesium aluminum Silicate | 2.0 |
| Methyl paraben | 0.18 |
| Propyl Paraben | 0.02 |
| Sorbitol Solution | 20.0 |
| Propylene Glycol | 20.0 |
| Purified Water | 54.5 |

Composition For Transdermal Delivery

The resulting composition is useful for administration in accordance with the methods of the present invention for inhibiting, i.e. treating or reducing the risk of, osteoarthritis lesions in a mammal in need thereof.

Similarly, a composition comprising other relative weights of alendronate, on an alendronic acid active weight basis are prepared. Also, compositions containing other bisphosphonates at appropriate active levels are similarly prepared: e.g., cimadronate, ibandronate, neridronate, olpandronate, risedronate, piridronate, pamidronate, zolendronate, and pharmaceutically acceptable salts thereof. In addition, compositions containing combinations of bisphosphonates are similarly prepared.

| Ingredient | Percent W/V |
|---|---|
| Alendronate Monosodium Trihydrate | 1.3% w/v |
| Butylated Hydroxyanisole | 0.02 |
| Polysorbate 80 | 3.0 |
| Diethyleneglycol monobutyl ether | 5.0 |
| n-Methylpyrrolidone | 90.7 |

Composition For Transdermal Delivery (Skin Patch)

The resulting composition is useful for administration in accordance with the methods of the present invention for inhibiting, i.e. treating or reducing the risk of, osteoarthritis lesions in a mammal in need thereof.

Similarly, compositions comprising other relative weights of alendronate, on an alendronic acid active weight basis are prepared. Also, compositions containing other bisphosphonates at appropriate active levels are similarly prepared: e.g., cimadronate, ibandronate, neridronate, olpandronate, risedronate, piridronate, pamidronate, zolendronate, and pharmaceutically acceptable salts thereof. In addition, compositions containing combinations of bisphosphonates are similarly prepared.

| Ingredient | Percent W/W |
|---|---|
| Alendronate Base | 5.0% w/w |
| Alcohol | 15.0 |
| Hydoxypropylcellulose | 1.0 |
| Mineral oil | 0.2 |
| Polyisobutylene | QSAD |
| Ethylenevinyl acetate | QSAD |

Injectables (IV/IM,SC/IP)

The resulting injectables are useful for administration in accordance with the methods of the present invention for inhibiting, i.e. treating or reducing the risk of osteoarthritis lesions in a mammal in need thereof.

Similarly, injectables comprising other relative weights of alendronate, on an alendronic acid active weight basis are prepared.

Also, injectables containing other bisphosphonates at appropriate active levels are similarly prepared: e.g., cimadronate, ibandronate, neridronate, olpandronate, risedronate, piridronate, pamidronate, zolendronate, and pharmaceutically acceptable salts thereof. In addition, injectables containing combinations of bisphosphonates are similarly prepared.

| Ingredient | Percent W/V |
|---|---|
| Alendronate Monosodium Trihydrate | 2.0% w/v |
| Sodium Citrate | 0.5 |
| Benzyl Alcohol | 2.0 |
| Edetate Sodium | 0.01 |
| Sodium Metabisulfite | 0.02 |
| Water for Injection | 95.5 |

Compositions for Intra-Nasal Delivery

The resulting composition is useful for administration in accordance with the methods of the present invention for inhibiting, i.e. treating or reducing the risk of, osteoarthritis lesions in a mammal in need thereof.

Similarly, compositions comprising other relative weights of alendronate, on an alendronic acid active weight basis are prepared. Also, compositions containing other bisphosphonates at appropriate active levels are similarly prepared: e.g., cimadronate, ibandronate, neridronate, olpandronate, risedronate, piridronate, pamidronate, zolendronate, and pharmaceutically acceptable salts thereof. In addition, compositions containing combinations of bisphosphonates are similarly prepared.

| Ingredient | Percent W/W |
|---|---|
| Alendronate Monosodium Trihydrate | 2.0% w/w |
| Carboxymethylcellulose sodium | 0.2 |
| Dextrose | 0.9 |
| Benzylalkonium chloride | 0.01 |
| Polysorbate 80 | 3.0 |
| Hydrochloric acid | 0.01 |
| Purified Water | 93.9 |

Sustained-Release Tablets

The resulting tablets are useful for administration in accordance with the methods of the present invention for inhibiting, i.e. treating or reducing the risk of, osteoarthritis lesions in a mammal in need thereof.

Similarly, tablets comprising other relative weights of alendronate, on an alendronic acid active weight basis are prepared. Also, tablets containing other bisphosphonates at appropriate active levels are similarly prepared: e.g., cimadronate, ibandronate, neridronate, olpandronate, risedronate, piridronate, pamidronate, zolendronate, and pharmaceutically acceptable salts thereof. In addition, tablets containing combinations of bisphosphonates are similarly prepared.

| Ingredient | Percent W/W |
|---|---|
| Alendronate Monosodium Trihydrate | 1.3% w/w |
| Citric Acid | 1.0 |
| Sodium Citrate | 0.5 |
| Cellulosic Polymer | 1.0 |
| Corn Starch | 5.0 |
| Sodium Starch Glycolate | 5.0 |
| Titanium Dioxide | 0.5 |
| Vanillin | 0.5 |
| Hydrogenated Castor Oil | 6.0 |
| Povidone | 5.0 |
| Acetylated Monoglycerides | 1.0 |
| Microcrystalline Cellulose | 18.0 |
| Lactose | 55.2 |

In addition to the ingredients exemplified above, formulations can also contain additional suitable buffers, colors, dispersants, flavors, stabilizers and preservatives as necessary.

What is claimed is:

1. A method for treating osteoarthritis in dogs resulting from joint instability associated with hip dysplasia or cruciate ligament damage in canines which comprises administering to the mammal a therapeutically effective amount of a bisphosphonate.

2. The method of claim 1 wherein the bisphosphonate is alendronate, cimadronate, clodronate, etidronate, ibandronate, incadronate, minodronate, neridronate, olpadronate, pamidronate, piridronate, risedronate, tiludronate, zolendronate, or a combination thereof.

3. The method of claim 2 wherein the bisphosphonate is alendronate or a pharmaceutically acceptable salt thereof.

4. A method for treating pain associated with osteoarthritis associated with hip dysplasia or cruciate ligament damage in canines which comprises administering to the mammal a therapeutically effective amount of a bisphosphonate.

5. The method of claim 4 wherein the bisphosphonate is alendronate, cimadronate, clodronate, etidronate, ibandronate, incadronate, minodronate, neridronate, olpadronate, pamidronate, piridronate, risedronate, tiludronate, zolendronate, or a combination thereof.

6. The method of claim 5 wherein the bisphosphonate is alendronate or a pharmaceutically acceptable salt thereof.

7. A method for reducing joint swelling in canines which comprises administering to the mammal a therapeutically effective amount of a bisphosphonate.

8. The method of claim 7 wherein the bisphosphonate is alendronate, cimadronate, clodronate, etidronate, ibandronate, incadronate, minodronate, neridronate, olpadronate, pamidronate, piridronate, risedronate, tiludronate, zolendronate, or a combination thereof.

9. The method of claim 8 wherein the bisphosphonate is alendronate or a pharmaceutically acceptable salt thereof.

10. A method for preventing the shallowing of the acetabulum in the hip of a canine which comprises administering to the mammal a therapeutically effective amount of a bisphosphonate.

11. The method of claim 10 wherein the bisphosphonate is alendronate, cimadronate, clodronate, etidronate, ibandronate, incadronate, minodronate, neridronate, olpadronate, pamidronate, piridronate, risedronate, tiludronate, zolendronate, or a combination thereof.

12. The method of claim 11 wherein the bisphosphonate is alendronate or a pharmaceutically acceptable salt thereof.

13. A method for preventing osteophyte formation in the hip or stifle of a canine which comprises administering to the mammal a therapeutically effective amount of a bisphosphonate.

14. The method of claim 13 wherein the bisphosphonate is alendronate, cimadronate, clodronate, etidronate, ibandronate, incadronate, minodronate, neridronate, olpadronate, pamidronate, piridronate, risedronate, tiludronate, zolendronate, or a combination thereof.

15. The method of claim 14 wherein the bisphosphonate is alendronate or a pharmaceutically acceptable salt thereof.

16. A method for treating osteoarthritis associated with hip dysplasia or cruciate ligament damage in canines which comprises administering to the mammal a therapeutically effective amount of a bisphosphonate and a nonsteroidal anti-inflammatory agent.

17. The method of claim 16 wherein the bisphosphonate is alendronate, cimadronate, clodronate, etidronate, ibandronate, incadronate, minodronate, neridronate, olpadronate, pamidronate, piridronate, risedronate, tiludronate, zolendronate, or a combination thereof; and the nonsteroidal anti-inflammatory agent is carprofen, etodolac, ibuprofen, ketoprofen, meloxicam, naproxen, celecoxib, deracoxib, etoricoxib, firocoxib, lumaricoxib, parecoxib, rofecoxib, or valdecoxib.

18. The method of claim 17 wherein the bisphosphonate is alendronate or a pharmaceutically acceptable salt thereof and the non-steroidal anti-inflammatory agent is rofecoxib.

19. The method of claim 17 wherein the bisphosphonate is alendronate or a pharmaceutically acceptable salt thereof and the non-steroidal anti-inflammatory agent is firocoxib.
